(19) United States
(12) Patent Application Publication
(10) Pub. No.: US 2005/0079594 A1
(43) Pub. Date: Apr. 14, 2005

(54) Method of removing a biofilm

(75) Inventor: Karine Marion, Saint Maurice De Beynost (FR)

Correspondence Address: OLIFF & BERRIDGE, PLC, P.O. BOX 19928, ALEXANDRIA, VA, 22320, US

(73) Assignees: Karine Marion, Saint Maurice De Beynost (FR); Thierry Sanchez, Saint Maurice De Beynost (FR)

(21) Appl. No.: 10/695,823

(22) Filed: Oct. 30, 2003

Related U.S. Application Data
(60) Provisional application No. 60/422,508, filed on Oct. 31, 2002.

(30) Foreign Application Priority Data
Oct. 31, 2002 (FR) 02.13963

(51) Int. Cl. C12S 9/00; C12N 9/50

(52) U.S. Cl. 435/196; 435/264; 510/393; 435/201; 435/219

(57) ABSTRACT

The present invention relates to a method of removing a biofilm, which comprises at least the following steps, carried out simultaneously or consecutively: a) a solution comprising an enzyme mixture containing at least one enzyme chosen from the group of proteases, at least one enzyme chosen from the group of esterases and an amylase is prepared; b) a solution comprising a detergent with an alkaline pH is prepared; and c) said solutions are applied, by washing or by circulation, to the surface to be treated. It also relates to a kit intended for removing a biofilm, which comprises the solutions defined above and the compositions comprising the said solutions.

### BACKGROUND OF THE INVENTION

The present invention relates to the field of the disinfection and decontamination of equipment and instruments having surfaces liable to act as support for the deposition of a biofilm.

### DESCRIPTION OF THE PRIOR ART

This equipment and/or these instruments are, for example, those used in the medical field, such as analytical instruments and any equipment such as reusable medical devices, such as dialysis generators, and also implants (ocular implants, heart valves) and prostheses. The equipment used in dentistry, even mucus membranes and teeth themselves, whether natural or prosthetic, are also liable to be the site of biofilm deposition. The equipment used in the food and/or pharmaceutical industry may also be cited, as well as air-conditioning plants and, more generally, any equipment in contact with an aqueous medium liable to contain bacteria in suspension.

The current major problem is the removal of the biofilm formed by the biomass attached to the surfaces of the equipment, instruments and/or mucus membranes, which biofilm constitutes a cause of persistent infections and/or of contamination. This is because any bacterium in suspension in an aqueous medium has the property of adhering to the supports that it encounters in order to form a biofilm. This biofilm is an agglomerate of bacteria on a surface, these bacteria being surrounded by a matrix of exopolysaccharides; formation of the biofilm is a natural phenomenon. Once the biofilm has been formed, it is very difficult to remove.

Current decontamination and/or disinfection methods that have been proposed for combating biofilms, although having a certain degree of effectiveness especially from the antibacterial standpoint, do not, however, remove the biofilm from the support, thereby encouraging its redevelopment and, in the particular case of hemodialysis, leaves supports for pyrogens on the surface.

In certain instruments or equipment, the biofilm is also enhanced by deposits of scale formed from calcium carbonate or magnesium carbonate.

The current use of decalcifying solutions to enhance the action of purely disinfecting solutions for the treatment of certain equipment does not, however, completely remove this biofilm except by using products such as bleach, which products, although effective against the biofilm, can often destroy the medical instruments and equipment. Furthermore, these solutions cannot be used on surfaces in contact with tissue and/or directly on mucus membranes.

L. F. Jacquelin, Pathologie Biologie, May 1994, page 425, teaches the sequential use of enzymes and of a phenolic disinfectant for the destruction of biofilms. C. Johansen, Applied and Environmental Microbiology, September 1997, page 3724, teaches the use of enzyme combinations such as glucose oxydases and lactoperoxydase. WO 01/53010 teaches an enzymatic method of removing biofilms, comprising the use of an enzyme belonging to the group of carbohydrases and proteases and their sequential use and also in combination with, or independently of, agents belonging to the group of biocides, chelating agents and other cleaning agents. Also taught, in EP 1 186 574, is a method of removing biofilms from surfaces in contact with water, characterized in that it includes cleaning with an enzymatic active principle and cleaning with a disinfectant intended to kill the bacteria released by the action of the enzyme mixture, but the results obtained are unsatisfactory and, at the present time, there is no process or product able to remove these biofilms.

The present invention allows the problem to be solved by the implementation of a method and by a selection of products making it possible to obtain a level of removal effectiveness never hitherto achieved.

### SUMMARY OF THE INVENTION

The present invention relates to a method of removing a biofilm, which comprises at least the following steps, carried out simultaneously or consecutively:
a) a solution comprising an enzyme mixture containing at least one enzyme chosen from the group of proteases, at least one enzyme chosen from the group of esterases and an amylase is prepared;
b) a solution comprising a detergent with an alkaline pH is prepared; and
c) said solutions are applied, by washing or by circulation, to the surface to be treated.

In a variant, the method according to the invention furthermore comprises the following steps, carried out simultaneously or consecutively:
d) a solution comprising an acid capable of dissolving deposits of mineral salts is prepared; and
e) said solution is applied, by washing or by circulation, to the surface to be treated.

The invention also relates to:
the method according to the invention, wherein the enzyme chosen from the group of proteases is chosen from the group formed by exopeptidases or endopeptidases, such as trypsin;
the method according to the invention, wherein the enzyme chosen from the group of esterases is a carboxyl ester hydrolase, such as lipase, a phospholipase and/or a phosphonodiesterase, such as ribonuclease;
the method according to the invention, wherein the enzyme mixture furthermore comprises an enzyme chosen from the group formed by osidases or carbohydrases such as glycosidase and galactosidase;
the method according to the invention, wherein the enzyme mixture is pancreatin;
the method according to the invention, wherein the detergent is an alkaline solution containing surfactants;
the method according to the invention, wherein the detergent is an alkaline solution containing surfactants and a quaternary ammonium; and
the method according to the invention, wherein the detergent furthermore contains a disinfectant such as a sodium hypochlorite or potassium hypochlorite solution.

When, in a variant, the method comprises a step of washing with an acid solution in order to remove the deposits of mineral salts, the acid is chosen from the group formed by citric acid, peractetic acid, glycolic acid and hydroxyacetic acid.

The invention also relates to a kit intended for removing a biofilm, which comprises at least one solution of an enzyme mixture containing at least one enzyme chosen from the group of proteases, at least one enzyme chosen from the group of esterases and an amylase, and at least one solution of a detergent with an alkaline pH.

The invention also relates to:
a kit according to the invention, wherein the enzyme chosen from the group of proteases is chosen from the group formed by exopeptidases or endopeptidases, such as trypsin;
a kit according to the invention, wherein the enzyme chosen from the group of esterases is a carboxyl ester hydrolase, such as lipase, a phospholipase and/or a phosphonodiesterase, such as ribonuclease;
a kit according to the invention wherein the enzyme mixture furthermore comprises an enzyme chosen from the group formed by osidases or carbohydrases such as glycosidase and galactosidase;
a kit according to the invention, wherein the enzyme mixture is pancreatin;
a kit according to the invention, wherein the detergent is an alkaline solution containing surfactants;
a kit according to the invention, wherein the detergent is an alkaline solution containing surfactants and a quaternary ammonium;
a kit according to the invention, which furthermore includes a solution of a disinfectant such as a sodium hypochlorite or potassium hypochlorite solution;
a kit according to the invention, which furthermore includes a solution of an acid capable of dissolving deposits of mineral salts such as calcium carbonate;
a kit according to the invention, wherein, in the acid solution for removing the deposits of mineral salts, the acid is chosen from the group formed by citric acid, peractetic acid, glycolic acid and hydroxyacetic acid.

The enzyme mixture is preferably used at a concentration of between 0.1 and 10% by weight/volume; the detergent is preferably used at a concentration of between 0.1 and 30% by volume/volume, and when the latter furthermore includes a disinfectant, the disinfectant is preferably added at a concentration of 0.01 to 2%.

The period of application according to the method of the invention of the solution comprising the enzyme mixture is between 5 min and one hour, depending on the type of biofilm, on how old it is and on the material; this application is preferably carried out at a temperature between room temperature and 40° C., preferably at 37° C.

The solution comprising a detergent with an alkaline pH is applied for a time that may vary from 5 min to 24 hours, at a temperature between room temperature and 90° C., depending on the type of biofilm and on the material treated.

The invention also relates to a composition intended for removing the biofilm, characterized in that it comprises an enzyme mixture containing at least one enzyme chosen from the group of proteases, at least one enzyme chosen from the group of esterases and an amylase, and a detergent with an alkaline pH.

More particularly, the composition according to the invention is characterized in that the enzyme mixture is pancreatin.

In a variant of the invention, the solution comprising the enzyme mixture and the solution comprising the detergent form a single solution; the invention then also relates to a composition intended for removing a biofilm, which method comprises an enzyme mixture containing at least one enzyme chosen from the group of proteases, at least one enzyme chosen from the group of esterases and an amylase, and a detergent with an alkaline pH.

In one particular embodiment, the method of the invention is one in which the detergent is a neutral solution or an acid solution containing surfactants; in this variant, when the detergent is an acid solution, it is possible, without an additional step, to dissolve mineral salts, when the phenomenon of scaling is very substantial.

### DESCRIPTION OF THE PREFERRED EMBODIMENTS

The term “biofilm” is understood to mean a set of microorganisms that have grown on a support, especially bacteria, viruses, parasites and fungi. This biofilm grows and the microorganisms secrete a matrix of exopolymers containing, inter alia, exopolysaccharides that will form a biological film called “slime” or “glycocalix” and is in the form of a gelatinous deposit on the surface of the walls.

The term “pancreatin” is understood to mean a pancreatic extract containing all of the digestive enzymes from the pancreas, especially proteolytic enzymes or proteases and hydrolases, especially esterases, such as lipase, amylase and ribonuclease, and trypsin. Reference will be made to the definition given in the European Pharmacopea.

The term “detergent” is understood to mean any product whose composition has been specially designed to develop detergency phenomena, and which comprises, as essential components, surface agents, which are surfactants, and optionally additional components (various adjuvants, reinforcing agents, fillers and additives). Surfactants are chemical compounds which, when introduced into a liquid, lower its surface tension, with the effect of increasing the wetting properties.

The term “alkaline pH” is understood to mean an aqueous solution with a pH that is greater than 7, and preferably, in the present invention, a pH that is greater than or equal to 9.

The term “removal of the biofilm” is understood to mean the detachment of the biofilm from its support.

The effectiveness of the method according to the invention was tested experimentally as described below.

The method was tested and implemented experimentally on five types of biofilm; biofilms 1, 2, 3 and 5 were obtained using an in vitro model that mimics the hemodialysis generator:
biofilm 1: enriched with nutrients for accelerated growth (3 days) moderate thickness (about 10⁵ CFU/cm²), very rich in slime;
biofilm 2: not enriched with nutrients having grown over 1 month, equivalent to those actually encountered in dialysis generators (about 10³ CFU/cm²) very rich in scale crystals;
biofilm 3: enriched with nutrients for accelerated growth (5 days), thick (about 10⁹ CFU/cm²) very rich in slime;
biofilm 4: specimen of tubing conveying water for hemodialysis, taken from a center, covered with a biofilm of about 10³ CFU/cm² but having grown over more than one year; and
biofilm 5: enriched with nutrients for accelerated growth, having grown in a “preventive” model over three weeks.
   Production of the In Vitro Model

A 250 ml reactor vessel was filled with a nonsterile dialysate, prepared by diluting sterile apyrogenic hemodialysis concentrates (Clearflex® from Bieffe Medical) with nonsterile osmosed water containing Pseudomonas putida, Pseudomonas fluorescens and Flavimonas orizibitans, produced continuously in the laboratory.

The contaminating medium was circulated in a closed circuit in a loop of silicon tubing 1.5 meters in length and 5 mm inside diameter with a flow rate of 500 ml/min by a peristaltique pump. All the tubing and the reactor vessel were sterilized beforehand in an autoclave at 121° C. for 30 minutes. Thus, the dialysate naturally contaminated by the bacteria in the water was the only source of microorganisms.

The entire system was maintained at a temperature of 37° C. by a hotplate on which the reactor vessel was placed.

In the case of biofilms 1, 3 and 5, the bacterial growth and consequently the development of the biofilm were accelerated by adding an LB culture medium diluted 50-fold, i.e. an LB culture medium diluted to 5-fold and topped up with a flow equal to 1/10^{th} of that of the dialysate. The dialysate and culture medium top-up flow rates, regulated by a peristaltic pump, were 5 and 0.5 ml/minute, respectively.

In the case of biofilm 5, the model was modified in the following manner:

A nonsterile dialysate enriched with culture medium was made to flow for four hours through silicon tubing segments connected together by polypropylene couplers. Every four hours, the tubings were disconnected and integrated into disinfection systems (see below). After treatment, the tubings were reconnected and the contaminating medium resumed circulation for four hours. In parallel, control tubings that had never undergone disinfection were distributed in the circuit. Each day, two hemodialysis sessions, each interrupted by a disinfection session were thus able to be carried out. Overnight and at the weekends, the system was stopped after the last disinfection and the tubings were kept empty at room temperature. The system operated until a mature biofilm had developed on the control tubings.

Products and Combinations Tested

Seventeen products belonging to six different families were tested. The list of these products is given in table I. These produce were evaluated singly or in combinations. Thus, a complete screening of sixty combinations was carried out on biofilm 1; nine combinations were then evaluated on biofilm 2; finally, the best combination selected was tested on biofilms 3, 4 and 5.

TABLE I
List of Products Tested

| Family | Product | Supplier |
|---|---|---|
| Surfactants/detergents | Sodium dodecyl | Sigma |
| | sulfate | |
| | Triton | Sigma |
| | RBS | Chemical |
| | | products |
| | Tween | Sigma |
| Enzymes | Trypsin | Sigma |
| | Pancreatin | Sigma |
| | Fungal protease | Sigma |
| | Thermolysin | Sigma |
| Acids | Perchloric acid | Merck |
| | Citric acid | Merck |
| | Trichloroacetic | Merck |
| | acid | |
| Cell dissociation | Versene | Sigma |
| products | Cell | Sigma |
| | dissociation | |
| Alkalis | NaOH | Prolabo |
| | KOH | Prolabo |
| Miscellaneous | Bleach | — |
| | pH10 buffer | Prepared in the |
| | (bicarbonate) | lab |

Sampling

The tubings covered with biofilms 1, 2 and 3 were cut into segments 5 cm in length. For screening on biofilms 1 and 2, each segment for undergoing one of the various treatments to be investigated was selected by drawing lots. Control specimens taken at random from the silicon loop were kept untreated.

Treatment of the Specimens

The tubing segments to be treated were attached to the descending branch of a set-up consisting of two tubings—one ascending and the other descending, a peristaltic pump and a water bath (for treatments carried out a temperature above 20° C.). The product to be tested in “recirculation” mode was dissolved in a 100 ml flask and driven by the peristaltic pump at a rate of 500 ml/min in a closed circuit through the tubings for a duration corresponding to the contact times given in table II. The product to be tested in “static” mode was dissolved in a 100 ml flask and driven by the peristaltic pump until the tubings had been filled; the pump was then stopped and the product left in stasis for the desired contact time. After each treatment by a given product, the tubing specimens were rinsed for five minutes with osmosed water.

Methods of Evaluating the Effectiveness of the Treatments:

Three Fundamental Parameters were Used to Evaluate the Effectiveness of the Treatments:
The reduction in area covered;
The reduction in number of culturable bacteria; and
The reduction in the level of endotoxins.

The screening on biofilms 1 and 2 only took the first parameter into account. The best combination adopted was then evaluated in depth as regards its effectiveness on bacterial mortality and endotoxin elimination.

Method of Quantifying the Area Covered:

The control and treated biofilms were stained:
either with a 0.25% crystal violet solution;
or with a Baclight® fluorochrome solution (Syto 9 and propidium iodide). The viable bacteria show up green while the dead bacteria show up yellow or red.

The silicon tubing specimens covered with stained biofilms were attached to glass slides and observed under an optical microscope, the microscope being connected to a camera and to “Scion Images” image analysis software. Thus, several (6 to 10) photographs of the same specimen were taken, the stained area was evaluated quantitatively by the image analysis software and a mean covered area value per specimen was calculated. This mean value was compared with the covered area of the untreated control specimens: a percentage reduction in covered area was then calculated.

Moreover, for more accurate examination, the specimens treated by the most effective combination were observed under a laser confocal microscope.

Method of Quantifying the Culturable Bacteria:

The biofilm covering the tubing specimens was detached from the substrate using a mechanical scraper, ensuring complete, uniform and reproducible detachment. This scraper consisted of a power screwdriver at the end of which a flame-sterilizable stainless steel spatula was fixed. By rotating the spatula in the lumen of the tubing, the biomass was driven to the bottom of a sterile tube. This action was facilitated by a stream of sterile water. Any bacterial aggregates were then separated via the needle of a syringe. The number of culturable bacteria was determined by counting the CFUs after plating the resulting bacterial suspension on R₂A agar and incubating at room temperature for 7 days.

More precisely, the specimens proving to be uncontaminated after plating were completely filtered and the filtration membrane was incubated on R₂A agar at room temperature for 7 days.

Endotoxin Assay Method:

The bacterial endotoxins were quantified in the bacterial suspension resulting from the detachment (see above) by a standardized reference test, namely the kinetic chromogenic LAL test (Charles River Endosafe).

Results of the Study on Biofilms 1 and 2:

The results of the screening on biofilms 1 and 2 are shown in tables II and III.

TABLE II
Screening on biofilm 1

| Treat. | | | | | | |
|---|---|---|---|---|---|---|
| No. | Product | Conc. | ° C. | Time | Mode | Result |
| 1 | SDS | 5% | RT | 40 min | Stat | ** |
| 2 | Triton | 5% | RT | 40 min | Stat | * |
| 3 | RBS | 5% | RT | 40 min | Stat | ** |
| 4 | Tween | 5% | RT | 40 min | Stat | . |
| 5 | Versene | Pure | RT | 40 min | Stat | . |
| 6 | Trypsin | EDTA IX | RT | 40 min | Stat | . |
| 7 | Trypsin | 0.25% | 37 | 40 min | Stat | ** |
| 8 | SDS | 5% | RT | 40 min | Recirc | */. |
| 9 | Tween | 5% | RT | 40 min | Recirc | . |
| 10 | RBS | 5% | RT | 40 min | Recirc | *** |
| 11 | Perchlo ac | 0.05% | RT | 40 min | Stat | . |
| 12 | NaOH | 0.01N | RT | 40 min | Stat | * |
| 13 | KOH | 0.02N | RT | 40 min | Stat | ** |
| 14 | TCA | 0.25% | RT | 40 min | Stat | . |
| 15 | KOH | 0.02N | RT | 40 min | Recirc | ** |
| 16 | Triton | 5% | RT | 40 min | Recirc | . |
| 17 | RBS | 5% | RT | 1 h | Recirc | *** |
| 18 | RBS | 5% | RT | 24 h | Recirc | **** |
| 19 | RBS | 2% | RT | 1 h | Recirc | *** |
| 20 | RBS | 2% | RT | 24 h | Recirc | **** |
| 21 | KOH | 0.02N | RT | 1 h | Recirc | *** |
| 22 | KOH | 0.02N | RT | 24 h | Recirc | **** |
| 23 | Pancreatin | 1% | 37 | 1 h | Recirc | **** |
| 24 | Pancreatin | 1% | 37 | 1 h | Stat | ** |
| 25 | Pancreatin | 0.10% | 37 | 2 h | Recirc | . |
| 26 | Citric acid | 3% | RT | 40 min | Recirc | . |
| 27 | KOH | 0.002N | RT | 40 min | Recirc | ** |
| 28 | Cell dissoc | Pure | 37 | 40 min | Stat | ** |
| 29 | Trypsin | 0.25% | 37 | 40 min | Recirc | . |
| 30 | Protease | 0.25% | 37 | 40 min | Recirc | ** |
| 31 | RBS | 2% | RT | 40 min | Recirc | **** |
| 32 | RBS + Cl | 2% + 0.2% | RT | 40 min | Recirc | **** |
| 33 | Thermolys | 2 mg/50 ml | 37 | 40 min | Recirc | ** |
| 34 | Pancreatin | 0.50% | 37 | 40 min | Recirc | *** |
| 35 | KOH | 0.001N | RT | 40 min | Recirc | ** |
| 36 | RBS | 2% pH7 | RT | 40 min | Recirc | *** |
| 37 | Trypsin | 1% | 37 | 40 min | Recirc | * |
| 38 | Protease | 1% | 37 | 40 min | Recirc | ** |
| 39 | RBS | 1% pH10 | RT | 40 min | Recirc | **** |
| 40 | RBS | 0.5% | RT | 40 min | Recirc | *** (*) |
| | | pH10 | | | | |
| 41 | RBS | 1% pH10 | RT | 5 min | Recirc | *** |
| 42 | RBS | 0.1% | RT | 40 min | Recirc | ** (*) |
| | | pH10 | | | | |
| 43 | RBS + | 1% pH10 | 40 | 5 min | Recirc | * |
| | Pancreatin | 1% | | | | |
| 44 | RBS + | 1% pH10 | 40 | 5 min | Recirc | ** |
| | Pancreatin | 0.50% | | | | |
| 45 | RBS + | 1% pH10 | 40 | 40 | Recirc | *** |
| | Pancreatin | 0.50% | | | | |
| 46 | pH10 Buffer | Pure | 37 | 40 | Recirc | * |
| 47 | Pancreatin | 0.5 pH10 | 37 | 40 | Recirc | ** |
| 48 | Pancreatin | 0.5% pH7 | 37 | 5 min | Recirc | ***** |
| | and RBS | 1% pH10 | RT | 5 min | Recirc | |
| 49 | Pancreatin | 0.25% | 37 | 5 min | Recirc | *** |
| | | pH7 | | | | |
| 50 | Pancreatin + | 0.25% | 37 | 5 min | Recirc | ** |
| | Thermolys | pH7 | | | | |
| | | 2 mg/50 ml | | | | |
| 51 | Pancreatin + | 0.25% | 37 | 5 min | Recirc | ** |
| | Thermolys + | pH7 | | | | |
| | Protease | 2 mg/50 ml | | | | |
| | | 0.25% | | | | |
| 52 | Pancreatin + | 0.25% | 37 | 5 min | Recirc | ** |
| | Thermolys + | pH7 | | | | |
| | Protease + | 2 mg/50 ml | | | | |
| | Trypsin | 0.25% | | | | |
| | | 0.25% | | | | |
| 53 | Pancreatin | 0.25 | 37 | 5 min | Recirc | ***** |
| | and RBS | 1% | RT | 5 min | | |
| 54 | Pancreatin | 0.25 | 37 | 5 min | Recirc | ***** |
| | and RBS | 0.50% | RT | 5 min | | |
| 55 | Pancreatin | 0.25% | 37 | 5 min | Recirc | ***** |
| | and RBS | 0.50% | RT | 30 min | | |
| 56 | Pancreatin | 0.10% | 37 | 5 min | Recirc | ***** |
| | and RBS | 0.50% | RT | 5 min | | |
| 57 | Pancreatin | 0.10% | 37 | 5 min | Recirc | ***** |
| | and RBS | 0.10% | RT | 5 min | | |
| 58 | Citric acid | 3% | 37 | 5 min | Recirc | ***** |
| | and RBS | 1% | RT | 30 min | | |
| 59 | Citric acid | 3% | 37 | 5 min | Recirc | ***** |
| | and | 0.50% | 37 | 5 min | | |
| | pancreatin | 1% | RT | 30 min | | |
| | and RBS | | | | | |
| 60 | Pancreatin | 0.50% | 37 | 5 min | Recirc | ***** |
| | and RBS | 1% | RT | 30 min | | |

| | | % covered area |
|---|---|---|
| Key | Removal | reduction |
| . | No removal | 0 |
| * | Poor removal | 0-25% |
| ** | Moderate removal | 25-50% |
| *** | Good removal | 50-75% |
| **** | Excellent removal | 75-99% |
| ***** | Complete removal | 100% |

+ = mixture;
and = sequential application

TABLE III
Screening on biofilm 2

| Treat. | | | | | | |
|---|---|---|---|---|---|---|
| No. | Product | Conc. | ° C. | Time | Mode | Result |
| 1 | Pancreatin | 0.5% pH7 | 37 | 5 min | Recirc | *** |
| | and RBS | 1% pH10 | RT | 5 min | Recirc | |
| 2 | Citric | 3% | 37 | 5 min | Recirc | ***** |
| | acid and | 1% | RT | 30 min | | |
| | RBS | | | | | |
| 3 | Pancreatin | 0.50% | 37 | 5 min | Recirc | ***** |
| | and citric | 3% | 37 | 5 min | | |
| | acid | 1% | RT | 30 min | | |
| | and RBS | | | | | |
| 4 | Pancreatin | 0.50% | 37 | 5 min | Recirc | *** |
| | and RBS | 1% | RT | 30 min | | |
| 5 | Citric | 3% | 37 | 5 min | Recirc | ***** |
| | acid and | 0.50% | 37 | 5 min | | |
| | pancreatin | 1% | RT | 30 min | | |
| | and RBS | | | | | |
| 6 | Pancreatin | 0.50% | 37 | 5 min | Recirc | *** |
| | and RBS | 1% | RT | 30 min | | |
| 7 | Citric | 3% | 37 | 5 min | Recirc | **** |
| | acid | | | | | |
| 8 | Citric | 3% | 37 | 5 min | Recirc | ***** |
| | acid and | 0.50% | RT | 30 min | | |
| | RBS | | | | | |
| 9 | Citric | 3% | 37 | 5 min | Recirc | **** |
| | acid and | 0.10% | RT | 30 min | | |
| | RBS | | | | | |

The combination adopted following these screening tests was that which gave the best removal of the two biofilms, namely the following “combination K”:
Product A=pancreatin®, a laboratory reactant sold by Sigma: Pig pancreas extract, namely an enzyme mixture containing, inter alia, lipase, protease, amylase, trypsin, ribonuclease, etc. (see European Pharmacopea).
Product B=citric acid

In the particular case of disinfecting dialysis generators, this product acts as a decalcifier and removes the scale crystals that trap the bacteria and promote adhesion of the biofilm to the substrate.

Product C=RBS®, a foaming alkaline detergent solution, sold by Chemical Products, exhibiting bactericidal, virucidal and fungicidal properties, containing surfactants and a quaternary ammonium (disinfectant).

Qualitative and Quantitative Data:

Photographs of biofilms 1 and 2 before and after the action of combination K have allowed the action of the combination to be visually quantified.

Table IV gives the values of the parameters measured before and after action of combination K.

TABLES IV
Quantitative data for evaluating the
effectiveness of combination K on biofilms 1 and 2

| | | | |
|---|---|---|---|
| | Before | After | |
| Parameter | treatment | treatment | % reduction |

Table IVa) Biofilm 1

| | | | |
|---|---|---|---|
| Area covered | 20 | <0.001 | >99.99 |
| (sq.in) | | | |
| Culturable | 10⁵ | <1 | >99.999 |
| bacteria | | | |
| (CFU/cm²) | | | |
| Endotoxins | 10039 | <0.005 | >99.99 |
| (EU/cm²) | | | |

Table IVb) Biofilm 2

| | | | |
|---|---|---|---|
| Area covered | 13.4 | <0.001 | >99.99 |
| (sq.in) | | | |
| Culturable | 3 × 10³ | <1 | >99.999 |
| bacteria | | | |
| (CFU/cm²) | | | |
| Endotoxins | 40 | <0.005 | >99.99 |
| (EU/cm²) | | | |

Determination of the MIC of RBS:

Since the anitbacterial capacity of this combination is provided by RBS, its minimal inhibitory concentration was determined on the microorganisms constituting the biofilms under examination.

A mixture of contaminated fresh dialysate (prepared with nonsterile osmosed water containing the microorganisms described above) and LB medium in the proportions of 50/50 v/v was prepared. RBS solutions with concentrations of 100%, 50%, 10%, 5%, 1%, 0.5% and 0.1% were produced by cascade dilutions, and then 300 μl of each of these solutions were added to 3 ml of the contaminated mixture. After incubation for 12 hours at room temperature, the CFUs were counted on R₂A agar for each of the RBS concentrations tested.

The MIC is defined as being the lowest concentration that inhibits the growth of microorganisms. The results are given in table V.

TABLE V
Determination of the MIC of the RBS

| | Conc (%) | | | | | | | | | |
|---|---|---|---|---|---|---|---|---|---|---|
| | 0 | 0.01 | 0.05 | 0.5 | 1 | 2 | 3 | 4 | 5 | 10 |
| UFC/ml | 2 × 10⁸ | 4.8 × 10⁷ | 2.1 × 10⁷ | 9 × 10⁶ | 6.1 × 10⁶ | 1.4 × 10⁵ | 1200 | 200 | 0 | 0 |

For safety, the choice was made to use the RBS solution in dilute form so as to obtain an MIC of 1.5.

Initial Protocol Adopted:

0.5%/pH 7.3 pancreatin: preparation for 100 ml: 500 mg of pancreatin powder+1 g of powdered PBS (phosphate buffer saline) buffer (Sigma), diluted in 100 ml of Hemodialysis Water (HDW).

Closed-circuit flow of the solution in the tubings with a flow rate of 500 ml/min for 5 minutes at 37° C.

Rinsing: 5 minutes with HDW at 500 ml/min in open circuit.

3%/pH 2.2 citric acid: preparation for 100 ml: 3 g of powdered citric acid (Merck) in 100 ml of HDW.

Closed-circuit flow of the solution in the tubings with a flow rate of 500 ml/min for 5 minutes at 20° C.

Rinsing: 5 minutes with HDW at 500 ml/min in open circuit.

7%/pH 10 RBS®: preparation for 100 ml: 7 ml of concentrated solution+566 mg of powdered sodium carbonate+388 mg of powdered sodium bicarbonate, diluted with HDW to make up to 100 ml.

Closed-circuit flow of the solution in the tubings with a flow rate of 500 ml/min for 30 minutes at 20° C.

Rinsing: 5 minutes with HDW at 500 ml/min in open circuit.

Results of the Study of Biofilms 3 and 4:

Combination K in its initial form described above left a few cells adhering to biofilms 3 and 4, particularly thick or old biofilms. To remove such biofilms, an “enriched formula” of combination K was developed.

“Enriched” Formula:

1%/pH 7.3 pancreatin: preparation for 100 ml: 1 g of pancreatin powder+1 g of powdered PBS (phosphate buffer saline) buffer (Sigma), diluted in 100 ml of hemodialysis water (HDW).

Closed-circuit flow of the solution in the tubings with a flow rate of 500 ml/min for 5 minutes at 37° C.

Rinsing: 5 minutes with HDW at 500 ml/min in open circuit.

5%/pH 2.2 citric acid: preparation for 100 ml: 5 g of powdered citric acid (Merck) in 100 ml of HDW. Closed-circuit flow of the solution in the tubings with a flow rate of 500 ml/min for 30 minutes at 20° C.

Rinsing: 5 minutes with HDW at 500 ml/min in open circuit.

15%/pH 10 RBS®: preparation for 100 ml: 15 ml of concentrated solution plus 566 mg of powdered sodium carbonate+388 mg of powdered sodium bicarbonate+6 ml of 5.2% concentration bleach (final sodium hypochlorite concentration: 0.3%), diluted in HDW to make up to 100 ml.

Closed-circuit flow of the solution in the tubings with a flow rate of 500 ml/min overnight at 20° C.

Rinsing: 5 minutes with HDW at 500 ml/min in open circuit.

Qualitative and Quantitative Data:

Photographs of biofilm 4 before and after the action of enriched combination K allowed the effectiveness of the method according to the invention to be visually quantified.

Table VI gives the values of the parameters measured before and after the action of enriched combination K on biofilm 4.

TABLE VI
Quantitative data for evaluating the
effectiveness of enriched combination K on biofilm 4

| | Before | After | |
|---|---|---|---|
| Parameter | treatment | treatment | % reduction |
| Area covered | 25 | <0.001 | >99.99 |
| (sq.in) | | | |
| Culturable | 1600 | <1 | >99.999 |
| bacteria | | | |
| (CFU/cm²) | | | |
| Endotoxins | 115 | <0.005 | >99.999 |
| (EU/cm²) | | | |

Results of the Study on Biofilm 5:

A very thick (more than 3×10⁹ CFU/cm²) biofilm very rich in slime, very rich in bacterial endotoxins and completely covering the surface of the specimen (30 square inches) grew on the surface of the untreated control specimens, whereas only a few adherent dead cells were deposited on the surface of the treated specimens, treated every 4 hours with unenriched combination K (initial formula).

The quantitative data are given in table VII.

TABLE VII
Effectiveness of combination K on biofilm 5

| | Without | With | |
|---|---|---|---|
| Parameter | treatment | treatment | % inhibition |
| Area covered | 30 | 1.3 | 96 |
| (sq.in) | | | |
| Culturable | 3.9 × 10⁹ | <1 | >99.999 |
| bacteria | | | |
| (CFU/cm²) | | | |
| Endotoxins | 65282 | 0.4 | >99.999 |
| (EU/cm²) | | | |

Photographs of the control biofilm and of the treated specimens allowed the effectiveness of the method according to the invention to be verified.

The method, the kit and the composition according to the invention can be used in the circuits of hemodialysis equipment to combat legionellosis for example in hot-water circuits and air-conditioning systems and cooling towers, in the agrifoodstuffs industry, in climate-controlled rooms or in confined-atmosphere rooms, for cleaning dentistry equipment, and for reusable and non-autoclavable medical instruments.

In fluid flow equipment, the method according to the invention will be employed by introducing the solution or solutions simultaneously or sequentially into the circuits from which the biofilm must be removed, by making said solution(s) circulate for a period sufficient to allow the biofilm to be removed, followed by purging and rinsing if necessary.

For treating surfaces, worksurfaces and prostheses, the method according to the invention will be carried out by application of or by immersion in the solution or solutions according to the invention, sequentially or simultaneously, followed if necessary by rinsing.

1. A method of removing a biofilm, which comprises at least the following steps, carried out simultaneously or consecutively:
a) a solution comprising an enzyme mixture containing at least one enzyme chosen from the group of proteases, at least one enzyme chosen from the group of esterases and an amylase is prepared;
b) a solution comprising a detergent with an alkaline pH is prepared; and
c) said solutions are applied, by washing or by circulation, to the surface to be treated.

2. The method as claimed in claim 1, which furthermore comprises the following steps, carried out simultaneously or consecutively:
d) a solution comprising an acid capable of dissolving deposits of mineral salts is prepared; and
e) said solution is applied, by washing or by circulation, to the surface to be treated.

3. The method as claimed in claim 1, wherein the enzyme chosen from the group of proteases is chosen from the group formed by exopeptidases or endopeptidases, such as trypsin.

4. The method as claimed in claim 1, wherein the enzyme chosen from the group of esteraes is a carboxyl ester hydrolase, such as lipase, a phospholipase and/or a phosphonodiesterase, such as ribonuclease.

5. The method as claimed in claim 1, wherein the enzyme mixture furthermore comprises an enzyme chosen from the group formed by osidases or carbohydrases, such as glycosidase and galactosidase.

6. The method as claimed in claim 1, wherein the enzyme mixture is pancreatin.

7. The method as claimed in claim 1, wherein the detergent is an alkaline solution containing surfactants.

8. The method as claimed in claim 1, wherein the detergent is an alkaline solution containing surfactants and a quaternary ammonium.

9. The method as claimed in claim 1, wherein the detergent solution furthermore contains a disinfectant such as a sodium hypochlorite or potassium hypochlorite solution.

10. The method as claimed in claim 2, wherein, in the acid for removing the deposits of mineral salts, the acid is chosen from the group formed by citric acid, peractetic acid, glycolic acid and hydroxyacetic acid.

11. A kit intended for removing a biofilm, which comprises at least one solution of an enzyme mixture containing at least one enzyme chosen from the group of proteases, at least one enzyme chosen from the group of esterases and an amylase, and which comprises at least one solution of a detergent with an alkaline pH.

12. The kit as claimed in claim 11, wherein the enzyme chosen from the group of proteases is chosen from the group formed by exopeptidases or endopeptidases, such as trypsin.

13. The kit as claimed in claim 11, wherein the enzyme chosen from the group of esterases is a carboxyl ester hydrolase, such as lipase, a phospholipase and/or a phosphonodiesterase, such as ribonuclease.

14. The kit as claimed in claim 11, wherein the enzyme mixture furthermore comprises an enzyme chosen from the group formed by osidases or carbohydrases, such as glycosidase and galactosidase.

15. The kit as claimed in claim 11, wherein the enzyme mixture is pancreatin.

16. The kit as claimed in claim 11, wherein the detergent is an alkaline solution containing surfactants.

17. The kit as claimed in claim 11, wherein the detergent is an alkaline solution containing surfactants and a quaternary ammonium.

18. The kit as claimed in claim 11, which furthermore includes a solution of a disinfectant such as a sodium hypochlorite or potassium hypochlorite solution.

19. The kit as claimed in claim 11, which furthermore includes a solution of an acid capable of dissolving deposits of mineral salts such as calcium carbonate.

20. The kit as claimed in claim 19, wherein the acid is chosen from the group formed by citric acid, peractetic acid, glycolic acid and hydroxyacetic acid.

21. A composition intended for removing a biofilm, which comprises an enzyme mixture containing at least one enzyme chosen from the group of proteases, at least one enzyme chosen from the group of esterases and an amylase, and a detergent with an alkaline pH.

22. The composition as claimed in claim 21, wherein the enzyme mixture is pancreatin.
